(19) United States
(12) Patent Application Publication
(10) Pub. No.: US 2019/0002492 A1
(43) Pub. Date: Jan. 3, 2019

(54) DERIVATIVES OF ALLOPREGNANOLONE AND OF EPIALLOPREGNANOLONE AND USES THEREOF FOR TREATING A NEUROPATHOLOGICAL CONDITION

(71) Applicants: Ayikoe Guy Mensah-Nyagan, Otswald (FR); Laurence Meyer, Strasbourg (FR); Christine Patte-Mensah, Otswald (FR); Omar Taleb, Strasbourg (FR); Michel Miesch, Herrlisheim (FR); Philippe Geoffroy, Strasbourg (FR); Blandine Ressault, Fondettes (FR)

(72) Inventors: Ayikoe Guy Mensah-Nyagan, Otswald (FR); Laurence Meyer, Strasbourg (FR); Christine Patte-Mensah, Otswald (FR); Omar Taleb, Strasbourg (FR); Michel Miesch, Herrlisheim (FR); Philippe Geoffroy, Strasbourg (FR); Blandine Ressault, Fondettes (FR)

(73) Assignees: UNIVERSITE DE STRASBOURG, Strasbourg (FR); CENTRE NATIONAL DE LA RECHERCHE SCIENTIFIQUE-CNRS, Paris Cedex (FR)

(21) Appl. No.: 15/973,645

(22) Filed: May 8, 2018

Related U.S. Application Data
(63) Continuation of application No. 14/006,842, filed on Oct. 3, 2013, now abandoned.

(30) Foreign Application Priority Data
Mar. 23, 2011 (FR) 1152390

(51) Int. Cl. C07J 7/00; C07J 71/00; C07J 13/00; C07J 17/00

(52) U.S. Cl. CPC C07J 7/002; C07J 17/00; C07J 13/005; C07J 71/0005

(57) ABSTRACT

The present invention relates to novel neurosteroids, especially derivatives of allopregnanolone and of epiallopregnanolone of formula (I) and the uses thereof as medicament for the treatment of neuropathologies, in particular neuropathies induced by the chemotherapy of a cancer. These molecules according to the invention have both preventative and curative effects. The neurosteroids according to the invention may also be of use in the treatment of neurodegenerative disorders, in particular for preventing neuronal cell death. They may thus be used as neuroprotectants and/or as an agent that stimulates neuronal proliferation.

### CROSS REFERENCE TO RELATED APPLICATIONS

This application is a continuation of U.S. application Ser. No. 14/006,842, filed on Oct. 3, 2013, which is a U.S. National Stage of PCT International Application No. PCT/FR2012/050616, filed on Mar. 23, 2012, which claims priority to FR Application No. 11523960, filed on Mar. 23, 2011, the disclosure of all of which is also incorporated herein by reference in their entirety.

### TECHNICAL FIELD

The invention relates to novel neurosteroids, in particular derivatives of allopregnanolone and of epiallopregnanolone, and uses thereof as medicaments for the treatment of neuropathologies, in particular neuropathies induced by chemotherapy for a cancer. These molecules may have both preventive and curative effects.

### BACKGROUND

One of the main side effects of cancer treatment, whether through chemotherapy or radiation therapy, is the induction of a painful peripheral neuropathy that generates discomfort in patients and seriously complicates medical care for cancer.

In particular, especially painful diffuse peripheral degeneration has been seen, for example caused by the use of certain cancer or antineoplastic medicaments such as, among others, vincristine, oxaliplatin and Taxol. The efficacy of these antineoplastics is therefore greatly limited by dose-dependent side effects, such as peripheral neuropathies causing intense pain in approximately 40% of patients.

In certain cases, the severity of the symptoms is such that it requires using less neurotoxic antineoplastics which are, however, less effective against cancer, or completely stopping chemotherapy, which considerably reduces the life expectancy of patients.

The anti-tumoral effect of antineoplastics is based on their ability to disrupt the polymerization of tubulin monomers into microtubules, which inhibits cell division. It has been suggested that the peripheral neuropathies caused by antineoplastics result from the toxic action on the microtubules of the spinal or cranial nerves, leading to deterioration of the axonal transport and axonal degeneration. Antineoplastic compounds also cause axonal demyelination responsible for ectopic electrical discharges, paresthesia or neuropathic pains.

Unfortunately, no effective prophylactic or curative treatment exists for the sensorimotor neuropathic symptoms caused by many antineoplastic medicaments used in chemotherapy.

It is therefore crucial to develop strategies to thwart the painful neuropathic side effects of antineoplastic medicaments to ensure patient comfort and improve the efficacy of treatments.

Thus, it is essential to identify neuroactive compounds capable of eliminating neuropathies, in particular the neuropathies caused by antineoplastic medicaments such as vincristine, oxaliplatin or Taxol, thereby contributing to improving antineoplastic therapies, in particular those based on the use of antineoplastics.

The article published in the journal Neurobiology of Disease 30, (2008), p 30-41 relates to the regulation of the pain threshold caused by a thermal or mechanical stimulus after a lesion of the sciatic nerve. In animals suffering from neuropathic pain caused by a lesion of the sciatic nerve, an intrathecal injection making it possible to increase the concentration of allopregnanolone or 3alpha,5alpha-THP (illustrated below) in the spinal cord causes a temporary analgesia in neuropathic rats. 3alpha,5alpha-THP is capable of causing that temporary analgesia because it potential lysis the central inhibition by activating the GABAergic system, GABA being the chief inhibitory neurotransmitter in the central nervous system of mammals.

Document WO09/05337 pertains to a treatment of migraine headaches based on steroid derivatives disubstituted in position 3, therefore different from the derivatives based on the invention or according to the invention, which are monosubstituted in position 3.

The neuroprotective and/or reparatory effects for the nervous tissue of the derivatives according to or in compliance with the invention, discovered by the Applicant and which may make it possible to counter the painful symptoms of peripheral neuropathies (allodynia and hyperalgesia) caused by a cancer therapy, are not mentioned in this document of the prior art.

The document entitled “3alpha-fluoro analogues of allopregnanolone and their binding to GABAa receptors”, collection of czechoslovak chemical communications, institute of organic chemistry & biochemistry, vol. 67, no 1, pages 30-46 specifically deals with derivatives of Allopregnanolone that interact with the type a GABAergic receptors and which modulate malfunctions related to the GABA. According to this document, the analogues of AP may modulate the cellular activity through a positive allosteric modulation of those receptors in the presence of GABA.

Document CZ20001888 relates to a molecule that acts specifically on the GABAa receptors and the pathologies associated with GABAergic signaling.

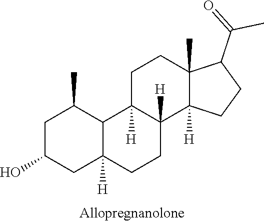

### SUMMARY

The principle of stimulating the central inhibition, via the GABAergic system, or reducing the neuronal excitability, via the opioid receptors as done by morphine and its derivatives, is the mode of action used by many antalgic medicaments that allow patients to no longer feel pain while the tissular and functional alterations responsible for nociceptive influxes persist. Once the neuroinhibiting activity of these traditional antalgics has ended, the painful symptoms reappear in the patient: this is then called temporary analgesia. These painful symptoms will continue as long as the tissular and functional damage has not been repaired. That is why many antalgics currently in use remain ineffective for the curative treatment of neuropathic pains most often resulting from traumatic, diffuse, cytotoxic lesions or a functional imbalance in the nervous structures involved in nociception. Consequently, neuropathic pain is a major public health issue, and the discovery of new strategies making it possible to permanently eradicate that pain is of tremendous medical and socioeconomic importance. To that end, it appears crucial to find treatments able to repair the damage or alterations to the nervous tissue that generate the noxious or abnormal signals evoking painful sensations. Only by repairing the tissular and neuronal damage will it be possible to restore normal sensory perception and permanently eliminate neuropathic pain.

The applicant has now found, quite surprisingly, derivatives of allopregnanolone and epiallopregnanolone that all share a neuroprotective function with varying degrees of efficacy, in particular the ability to repair the damaged nervous tissue and consequently eradicate the neuropathic pain definitively. Furthermore, the derivatives of allopregnanolone and epiallopregnanolone characterized by the applicant are also capable of protecting nervous cell cultures from death caused by oxidative stress, which emphasizes the enormous therapeutic potential of these derivatives for the treatment of neurodegenerative pathologies resulting from an anomaly in the oxidative metabolism.

In particular, the Applicant has found that the O-allyl derivatives of epiallopregnanolone, advantageously the 1-((3S,5S,10S,13S,17S)-3-(allyloxy)-10,13-dimethylhexadecahydro-1H-cyclopenta[a]phenanthren-17-yl)ethanone, have proven to be analgesics capable of definitively eliminating the painful symptoms, such as allodynia and hyperalgesia, for example caused by antineoplastics, without causing cell proliferation, which is contraindicated in cancer patients. Furthermore, owing to their ability to repair or protect against tissular and functional damage caused by antineoplastics on the peripheral nervous system, these neurosteroids exert a powerful neuroprotective effect.

Regarding the O-allyl derivatives of allopregnanolone, in particular the 1-((3R,5S,10S,13S,17S)-3-(allyloxy)-10,13-dimethylhexadecahydro-1H-cyclopenta[a]phenanthren-17-yl)ethanone, the 12-oxo-allopregnanolone derivatives, in particular the (3R,5S,10S,13S,17S)-17-acetyl-3-hydroxy-10,13-dimethyltetradecahydro-1H-cyclopenta[a]phenanthren-12(2H)-one and the 12-oxo-epiallopregnanolone derivatives, in particular the (3S,5S,10S,13S,17S)-17-acetyl-3-hydroxy-10,13-dimethyltetradecahydro-1H-cyclopenta[a]phenanthren-12(2H)-one, the Applicant has found that these are neuroprotectors that also cause cell proliferation. Consequently, these molecules are contraindicated to treat neuropathies caused by chemotherapy for cancer. However, they are absolutely indicated in noncancerous patients and may prove beneficial to treat neurodegenerative pathologies such as Alzheimer's disease, Parkinson's disease, Lou Gehrig's disease, etc. In fact, owing to their neuroproliferative activity, particularly high for 1-((3R.5S.10S.13S.17S)-3-(allyloxy)-10,13-dimethylhexadecahydro-1H-cyclopenta[a]phenanthren-17-yl)ethanone and (3R,5S,10S,13S,17S)-17-acetyl-3-hydroxy-10,13-dimethyltetradecahydro-1H-cyclopenta[a]phenanthren-12(2H)-one, these neurosteroids may stimulate or reactivate neurogenesis in the structures of the adult brain, such as the dentate gyrus of the hippocampus and the subventricular zones that contain neural stem cells so as to offset the neuronal losses observed in patients with such neurodegenerative diseases.

The present invention therefore relates to a derivative of allopregnanolone or epiallopregnanolone according to the following formula (I):

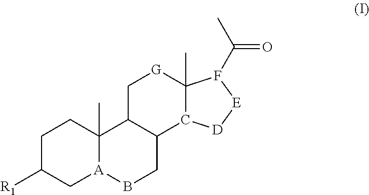

wherein
R¹ is a group chosen among the 3-alpha or 3-beta hydroxy groups, 3-alpha or 3-beta O-allyl groups, 3-alpha or 3-beta O-propargyl groups, 3-alpha or 3-beta O-glycol groups, 3-alpha or 3-beta O-PEG groups, 3-alpha or 3-beta O-glycol-allyl groups and 3-alpha or 3-beta O-PEG-allyl groups,
A is a carbon atom substituted by an atom chosen among 5-H alpha and 5-H beta and B is a methylene group; or
   A and B are carbon atoms forming a 5,6-double bond;
C is a carbon atom substituted by an atom chosen among 14-H alpha, 14-H beta, 14-alpha OH group and 14-beta OH and D is a methylene group; or
C and D are carbon atoms forming a 14,15-double bond;
F is a carbon atom substituted by an atom chosen among 17-H alpha and 17-H beta and E is a methylene group or a carbon atom substituted by a group chosen among 16-alkyl-alpha, 16-alkyl-beta, 16-OR²-alpha and 16-OR²-beta, with R² a group chosen among allyl, propargyl, glycol, PEG, glycol-allyl, PEG-allyl, or
F is a carbon atom substituted by a group chosen among 17-alkyl-alpha, 17-alkyl-beta, 17-OR²-alpha and 17-OR²-beta, with R² a group chosen among allyl, O-propargyl, glycol, PEG, glycol-allyl, PEG-allyl, and E is a methylene group or a carbon atom substituted by group chosen among 16-alkyl-alpha, 16-alkyl-beta, 16-OR²-alpha and 16-OR²-beta, with R² a group chosen among allyl, propargyl, glycol, PEG, glycol-allyl, PEG-allyl, or
E and F are involved in an epoxy cycle or a cyclopropyl and are chosen among 16,17-epoxy-alpha, 16,17-epoxy-beta, 16,17-methylene-alpha and 16,17-methylene-beta; or
E and F are carbon atoms forming a 16,17-double bond;
G is a carbonyl, a methylene or a carbon atom substituted by a 12-OR³-alpha or 12-OR³-beta group with R³ a H atom or a group chosen among the acetyl, alkyl and aryl groups, R³ being able to be chosen among ci-c6-alkyl, benzyl, p-methoxybenzyl, benzoyl, tigloyl, angeloyl, 2,2,2-trichloroethoxycarbonyl, o-aminobenzoyl, nicotinoyl, 2-methylbutyryl, isovaleryl, cinnamoyl, coumaroyl, o-hydroxybenzoyl and anthraniloyl,
   excluding (i) the molecule with formula BR053 below:

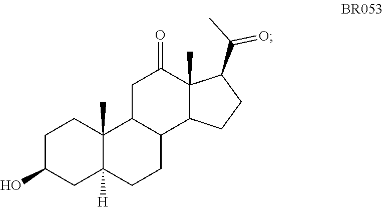 [SMILES: CC(=O)[C@H]1CCC2C3CC[C@H]4C[C@@H](O)CC[C@]4(C)C3CC(=O)[C@@]21C]

and
(ii) the molecule with formula BR338 below:

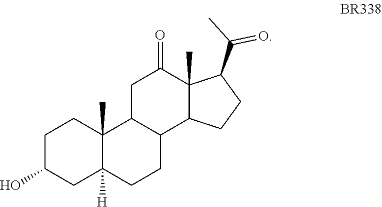 [SMILES: CC(=O)[C@H]1CCC2C3CC[C@H]4C[C@H](O)CC[C@]4(C)C3CC(=O)[C@@]21C]

According to one embodiment, this derivative is characterized in that:
R¹ is a group chosen among the 3-alpha or 3-beta O-allyl groups, the 3-alpha or 3-beta O-propargyl groups, the 3-alpha or 3-beta O-glycol groups, the 3-alpha or 3-beta O-PEG groups, the 3-alpha or 3-beta O-glycol-allyl groups and 3-alpha or 3-beta O-PEG-allyl groups,
A is a carbon atom substituted by an atom chosen among 5-H alpha and 5-H beta and B is a methylene group; or
A and B are carbon atoms forming a 5,6-double bond;
C is a carbon atom substituted by an atom chosen among 14-H alpha, 14-H beta, 14-alpha OH and 14-beta OH and D is a methylene group; or
C and D are carbon atoms forming a 14,15-double bond;
F is a carbon atom substituted by an atom chosen among 17-H alpha and 17-H beta, and E is a methylene group or a carbon atom substituted by a group chosen among 16-alkyl-alpha, 16-alkyl-beta, 16-OR²-alpha and 16-OR²-beta, with R² a group chosen among allyl, propargyl, glycol, PEG, glycol-allyl, PEG-allyl, or
F is a carbon atom substituted by a group chosen among 17-alkyl-alpha, 17-alkyl-beta, 17-OR²-alpha and 17-OR²-beta, with R² a group chosen among allyl, propargyl, glycol, PEG, glycol-allyl, PEG-allyl, and E is a methylene group or a carbon atom substituted by a group chosen among 16-alkyl-alpha, 16-alkyl-beta, 16-OR²-alpha and 16-OR²-beta, with R² a group chosen among allyl, propargyl, glycol, PEG, glycol-allyl, PEG-allyl, or
E and F are involved in an epoxy cycle or a cyclopropyl and are chosen among 16,17-epoxy-alpha, 16,17-epoxy-beta, 16,17-methylene-alpha and 16,17-methylene-beta; or
E and F are carbon atoms forming a 16,17-double bond; and
G is a carbonyl, a methylene or a carbon atom substituted by a 12-OR³ alpha or 12-OR³ beta group with R³ a H atom or a group chosen among the acetyl, alkyl and aryl groups, R³ being able to be chosen among ci-c6-alkyl, benzyl, p-methoxybenzyl, benzoyl, tigloyl, angeloyl, 2,2,2-trichloroethoxycarbonyl, o-aminobenzoyl, nicotinoyl, 2-methylbutyryl, isovaleryl, cinnamoyl, coumaroyl, o-hydroxybenzoyl and anthraniloyl.

### DETAIL DESCRIPTION

According to one embodiment, this derivative is characterized in that it involves a derivative of formula (1) in which R¹ is a group chosen among the 3-beta O-allyl, 3-beta O-propargyl, 3-beta O-glycol, 3-beta O-PEG, 3-beta O-glycol-allyl and 3-beta O-PEG-allyl groups and/or G is a methylene.

Derivatives according to the invention include:
the following derivative of formula BR297:

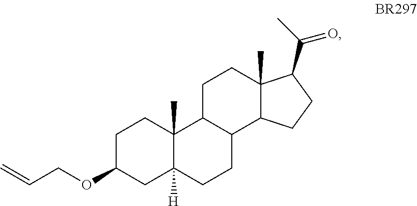 [SMILES: C=CCO[C@H]1CC[C@]2(C)C3CC[C@@]4(C)C(CC[C@@H]4C(C)=O)C3CC[C@H]2C1]

and
the following derivative of formula BR351:

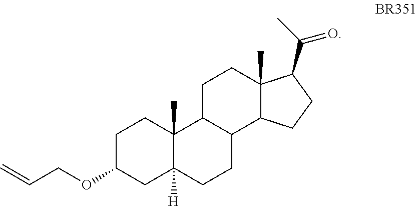 [SMILES: C=CCO[C@@H]1CC[C@]2(C)C3CC[C@@]4(C)C(CC[C@@H]4C(C)=O)C3CC[C@H]2C1]

The derivatives according to or in compliance with the invention, in particular compounds BR053, BR338, BR297 and BR351, may be used as a medicament. The invention therefore also relates to the use of derivatives according to or in compliance with the invention to obtain a medicament.

Thus, the derivatives according to the invention defined above or the derivative of formula BR338 or formula BR053, defined above, have proven useful as a neuroprotective agent and/or an agent stimulating the proliferation of nervous cells. These compounds in particular have proven useful to treat a neuropathology, that neuropathology being able to be chosen among the peripheral and central neural pathways, chronic pain, neurodegenerative diseases and sensory motor deficits caused by neuroinflammation. Preferably, the neuropathology is chosen among the peripheral and central neuropathies.

The neuropathology may be related to an allodynia or a hyperalgesia.

The neuropathy may be caused by an antineoplastic treatment, in particular a chemotherapeutic treatment using an antineoplastic for example chosen among spindle poisons, intercalating agents, topoisomerase DNA inhibitors, tyrosine kinase inhibitors, mTOR inhibitors, anti-angiogenic monoclonal antibodies, inhibitors of DNA synthesis and replication, and inhibitors of the polymerization or depolymerization of microtubules, still more advantageously chosen among vincristine, oxaliplatin, docetaxel, paclitaxel and the derivatives of Taxol in general.

In particular, the derivatives according to the invention defined above or in particular the derivatives of formula BR297 and BR351, defined above, or the derivatives of formulas BR338 and BR053, defined above, have proven useful as neuroprotectors, in particular as neurorepair agents. This neurorepair aspect is essential to effectively treat all of the pathologies caused by lesions of the nervous tissue.

The derivatives according to the invention, excluding BR297, and in particular the derivative of formula BR351 defined above or the derivatives of formula BR338 or formula BR053, defined above, are particularly useful as agents stimulating the proliferation of the nervous cells. This neuroproliferative aspect should be avoided in cancer treatment so as to avoid stimulating the proliferation of cancer cells. However, it is essential to overcome the neuronal cell deficit caused by a neurodegenerative pathology or by an accident with a lesion of healthy nervous tissue.

The derivatives according to the invention or the derivative of formula BR338 or formula BR053 are useful to prevent the cell death of neuroblastomas caused by oxidizing stress.

In the context of the present invention, it is meant by:
“neuroprotective effect”: (i) preventing the death of nerve cells following a cytotoxicity or a stress caused by endogenous or exogenous factors, (ii) favoring the repair of neurons and the nervous tissue in the case of a lesion, whether or not it is accidental, or caused by a curative treatment, such as chemotherapy, and/or (iii) reactivating neurogenesis in the mature nervous system or favoring the proliferation of nervous cells so as to overcome a cell deficit caused by a neurodegenerative disease.
“neuroprotective effect”: favoring the repair of neurons and the nervous tissue in the case of a lesion, whether or not it is accidental, or caused by a curative treatment, such as chemotherapy.
“neuroproliferative effect”: reactivating neurogenesis in the mature nervous system or favoring the proliferation of nervous cells so as to overcome a cell deficit caused by a neurodegenerative disease.
“anti-nociceptive effect”: blocking the transmission of noxious messages from the periphery toward the central nervous system or inhibiting the perception of the stimulations used to produce pain in healthy or pathological subjects.
“analgesic effect”: eliminating the pain existing in pathological subjects. The existence of pain is related to the etiological factors of the pathology.
“hyperalgesia”: a painful sensation of abnormally high intensity following a painful stimulation.
“allodynia”: a painful sensation caused by a stimulus that is not normally felt as pain.
“neuropathology”: all of the functional difficulties and tissular or cellular macroscopic or microscopic morphological alterations observed in diseases of the central and peripheral nervous systems. Some examples of neuropathologies are the peripheral and central neuropathies, chronic pain, neurodegenerative diseases (Alzheimer's disease, Lou Gehrig's disease, Huntington's disease, Parkinson's disease), neuroinflammatory and/or autoimmune pathologies (multiple sclerosis, autoimmune encephalitis).
“neuropathy”: all of the affections of the peripheral nervous system, i.e., the motor and sensory nerves of the limbs, as well as the nerves of the neurovegetative or autonomic nervous system controlling the activity of the organs and non-skeletal tissues. Peripheral nerve pathologies comprise, inter alia, polyneuritis, multiple neuritis or mononeuritis, chronic polyradiculoneuritis, Charcot-Marie-Tooth disease. In some cases, the lesions causing the sensory-motor and/or neurovegetative malfunctions characteristic of neuropathies are not located on the nerves (peripheral nervous system), but are found in the brain or spinal cord: these are then called central neuropathies.
“antineoplastic”: a medicament used to destroy cancerous cells or prevent their proliferation. Antineoplastics include: spindle poisons, intercalating agents, topoisomerase DNA inhibitors, tyrosine kinase inhibitors, mTOR inhibitors, antiangiogenic monoclonal antibodies, inhibitors of DNA synthesis and replication, and inhibitors of the polymerization or depolymerization of microtubules. Examples in particular include vincristine, oxaliplatin, docetaxel and paclitaxel.
“(Cₓ-Cₜ) group”: a group comprising between x and t carbon atoms.
“alkyl group”: a linear, branched or cyclic saturated aliphatic group, optionally substituted by a linear, branched or cyclic alkyl group. Examples include the methyl, ethyl, propyl, isopropyl, butyl, isobutyl, tertbutyl, cyclopropyl, cyclobutyl, cyclopentyl, cyclohexyl, methylcyclopropyl, cyclopropyl methyl groups.
“aryl group”: a mono or bicyclic aromatic group including from 6 to 10 atoms. Examples of aryl groups include phenyl and naphthyl.

The invention relates to derivatives of allopregnanolone and epiallopregnanolone.

The synthesis in particular of the derivatives of O-allyl epiallopregnanolone and O-allyl allopregnanolone is shown below in the following diagram 1. However, the synthesis of other derivatives according to the invention may easily be obtained by using, as starting compound, compounds that are easily synthesizable by one skilled in the art and/or commercially available.

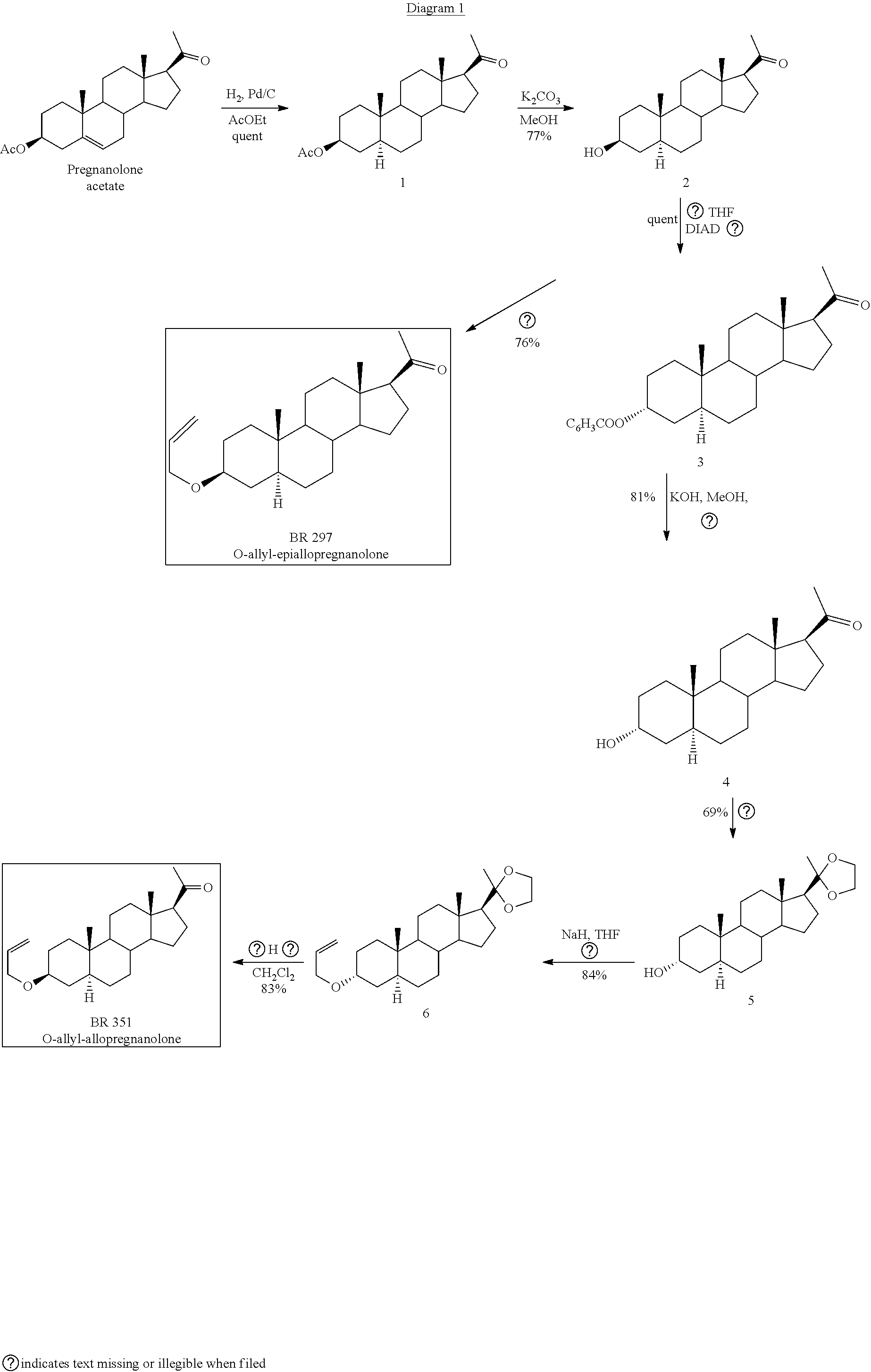

Diagram 1 above shows the different steps making it possible to obtain O-allyl epiallopregnanolone and O-allyl allopregnanolone from pregnanolone acetate.

First, the double bond of the commercially available pregnanolone acetate is reduced. A stereospecific hydrogenation of the 5,6-double bond of the pregnanolone acetate may be done in the presence of a catalytic quantity of Pd/C to provide compound 1. Then, a deprotection step of the 3 beta-acetate group occurs. This deprotection step may occur with potassium carbonate in methanol. At the end of this detection reaction, the epiallopregnanolone 2 is obtained. This epiallopregnanolone compound 2 can then be transformed into an O-allyl epiallopregnanolone derivative BR297. This reaction may be done by treating the epiallopregnanolone 2 with allyl bromide in the presence of Hunig's base (N, N-diisopropylethylamine) in dimethylformamide at reflux.

But epiallopregnanolone 2 may also be the source of the synthesis of another derivative of pregnanolone according to the invention: O-allyl allopregnanolone BR351.

In that case, the synthesis may be done according to the following reactions. The epiallopregnanolone 2 may be subject to a Mitsunobu reaction to form 3-alpha-benzoate-allopregnanolone 3. Following this, there is a deprotection reaction of the 3-alpha-benzoate with potassium hydroxide in methanol at reflux. This deprotection reaction yields the allopregnanolone compound 4. A protection reaction of the carbonyl group of said allopregnanolone 4 is then done. This reaction may for example be done with ethylene glycol in the presence of a catalytic quantity of p-TsOH. This protection reaction thus gives rise to the formation of a compound 5. This compound 5 may next undergo an allylation reaction by treatment with sodium hydride and allyl bromide in THF at reflux. One then obtains the protected O-allyl allopregnanolone 6 which, once deprotected, for example by acidified silica, leads to the O-allyl allopregnanolone compound BR351.

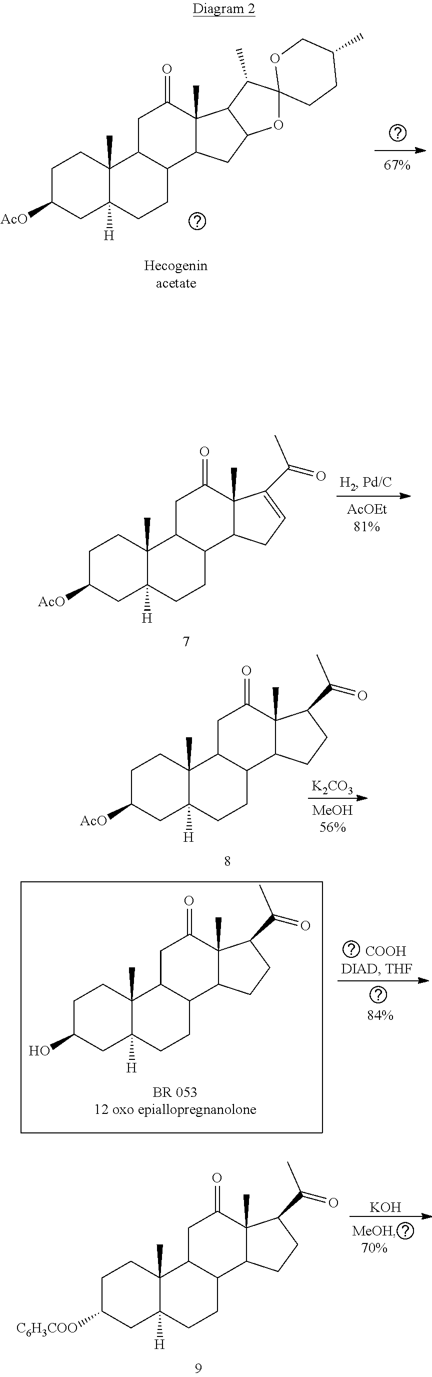

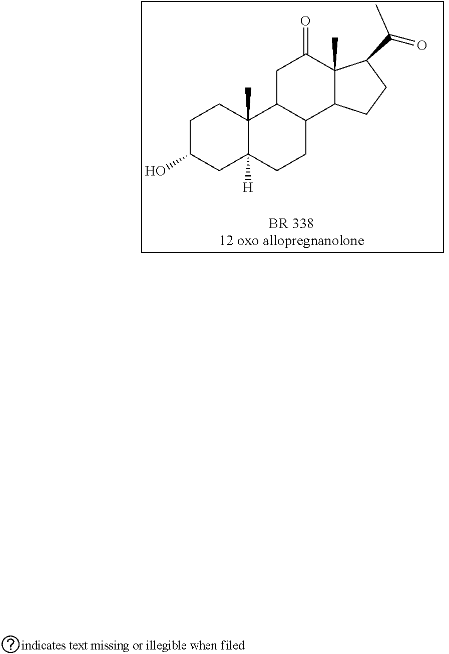 [SMILES: CC(=O)[C@H]1CCC2C3CC[C@H]4C[C@H](O)CC[C@]4(C)C3CC(=O)[C@@]21C]

Diagram 2 above shows different steps making it possible to obtain 12-oxo-epiallopregnanolone and 12-oxo-allopregnanolone from hecogenin acetate.

The synthesis of the 12-oxo-epiallopregnanolone compound BR053 may be done by transforming the dioxaspiro group of the commercially available hecogenin acetate. To that end, the hecogenin acetate is first treated in acetic anhydride by pyridine and in the presence of NH₄Cl. The product thus obtained is oxidized using chromium trioxide in acetic acid to yield compound 7 after heating in the acetic acid.

A hydrogenation of the enone 7 obtained followed by the deprotection of the 3 beta-acetate group that may be done with potassium carbonate in methanol leads to the 12-oxo-epiallopregnanolone compound BR053.

The 12-oxo-epiallopregnanolone compound BR053 may then be subjected to a Mitsunobu reaction leading to compound 9 which, after deprotection of the 3-alpha-benzoate group that may be done by potassium hydroxide in methanol at reflux, leads to the 12-oxo-allopregnanolone compound BR338.

The present Invention relates to derivatives of allopregnanolone or of epipregnanolone that primarily and clearly create a cellular effect that is completely independent of the GABAa receptors. In fact, electrophysiological experiments conducted on human neuroblastoma cells using a Patch-Clamp technique, in a Patch-Clamp whole cell configuration, made it possible to see that when the GABA is not in the pipette, i.e., not introduced, or when the GABAa receptors are not activated, said derivatives were capable of inducing a specific cellular activity. These results indicate that the derivatives according to the invention or in compliance with the invention interact with other ionic channels and different receptors from the GABAa receptors.

Malfunctions of the GABAa receptors are not the chief cause of the painful neuropathies caused by chemotherapy. The neurotoxic attack of antineoplastic medicaments creating painful neuropathies is primarily aimed against structural elements such as the cytoskeleton, the neurofilaments, the transporters, the calcium-dependent proteins of the spinal ganglion and/or axons of the peripheral nerves, as well as the key proteins in the production of the myelin sheath.

Consequently, all of the tests that pertain exclusively or primarily to the modulation of the GABAa receptors are ineffective in eradicating the painful neuropathies caused by antineoplastics.

Thus, the sophisticated properties of the derivatives according to the invention or in compliance with the invention, in particular BR297, constitute an original and powerful pharmacological means making it possible to effectively counter the painful neuropathic symptoms created by chemotherapy. In fact, BR297, which may essentially activate different cellular targets from those of the GABAa receptors in order to prevent or repair neuronal/axonal damage caused by antineoplastics, may also perform a partial allosteric modulation of the GABAergic system so as to reinforce its beneficial effects.

Molecules according to the invention or in compliance with the invention are effective as both a preventive and curative treatment, their main asset being their therapeutic capacity of the neuropathies instead of targeting, like the molecules of the prior art, only the nociceptive symptoms.

Other advantages may also appear to one skilled in the art upon reading the examples below. The following examples are provided as an illustration and are not limiting.

### EXAMPLES

The melting points were measured on a Stuart Scientific melting point device (SMP 3) and are not corrected. The reactions were done under argon with degassed solvents under magnetic agitation. The Et₂O and the THF were distilled in the presence of Na/benzophenone.

The thin layer chromatography (TLC) silica gel plates (Merck 60F254) and the spots were viewed under UV lamp (254 or 365 nm) and/or sprayed with a solution of phosphomolybdic acid (25 g of phosphomolybdic acid, 10 g of cerium sulfate, 60 ml of H₂SO4, 940 ml of H₂O) monitored on a hot plate while heating.

For column chromatography, silica gel (Merck Si 60 40-60 μm) was used.

IR spectrums were recorded on Bruker Alpha spectrophotometer (ATR, cm⁻¹). NMR spectroscopies ¹H were recorded at 300 MHz (Bruker AC-300) and NMR spectroscopies ¹³C at 75 MHz (Bruker AC-300) using the signal of the residual non-deuterated solvent as internal reference. Significant ¹H NMR data are tabulated in the following order: chemical displacement (δ) expressed in ppm, multiplicity (s, singlet; d, doublet; t, triplet; q, quartet; m, multiplet), coupling constants in hertz, number of protons.

High-resolution mass spectrometries (HRMS) were carried out in an Agilent 6520 Accurate Mass Q-TOF.

### Example 1: Synthesis of (3S,5S,10S,13S,17S)-17-acetyl-10,13-dimethylhexadecahydro-1, H-cyclopenta[alphenanthren-3-ylacetate 1

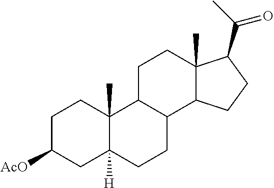 [SMILES: CC(=O)O[C@H]1CC[C@]2(C)C3CC[C@@]4(C)C(CC[C@@H]4C(C)=O)C3CC[C@H]2C1]

To a solution of pregnanolone acetate (5.14 g, 14.3 mmol) in AcOEt (125 ml), Pd/C at 10% (0.48 g) was added. The reaction mixture was ventilated under H₂ (vacuum/H₂) and was agitated vigorously under H₂ until absorption of 1.1 eq of H₂ (375 ml, 15.7 mmol). The reaction mixture was ventilated with Argon (vacuum/argon), the catalyst was filtered on Celite and washed with EtOH (100 ml), AcOEt (100 ml), CH₂Cl₂ (100 ml), then concentrated under reduced pressure. The solid obtained was purified by chromatography (100 g SiO₂, petroleum ether/ethyl acetate, 80/20) yielding compound 1 (4.98 g, 96%, F: 145.2-146.8° C.) with a solid white appearance.

IR (ATR): 1727, 1705.

¹H NMR (CDCh): 4.67 (1H, tt, J=5.0-11.3 Hz, H₃); 2.50 (1H, t, J=8.8 Hz, H₁₇); 2.20-1.90 (2H, m); 2.09 (3H, s, H₂₁); 2.00 (3H, s, OAc); 1.90-0.75 (19H, m); 0.80 (3H, s, Me); 0.68 (1H, td, J=4.2-12.0 Hz); 0.58 (3H, s, Me).

¹³C NMR (CDCl₃): 209.3 (C═O); 170.4 (C═O, OAc); 73.4 (CH, C₃); 63.7 (CH, Ci₇); 60.2 (CH₂); 56.5 (CH); 54.0 (CH); 44.5 (CH); 44.0 (CH); 38.9 (CH₂); 35.4 (CH); 35.4 (C); 33.9 (CH₂); 31.8 (CH₂); 28.4 (CH₂); 27.3 (CH₂); 24.3 (CH₂); 22.7 (CH₂); 21.1 (CH₂); 20.9 (CH₃); 14.1 (CH₃); 13.3 (CH₃); 12.1 (CH₃).

### Example 2: Synthesis of the 1-((3S,5S,10S,13S,17S)-3-hydroxy-10,13-dimethylhexadecahydro-1H cyclopenta[alphenanthren-17-yl)ethanone 2

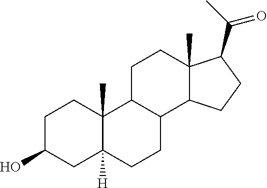 [SMILES: CC(=O)[C@H]1CCC2C3CC[C@H]4C[C@@H](O)CC[C@]4(C)C3CC[C@@]21C]

To a solution of compound 1 (4.82 g, 13.4 mmol) in a mixture of MeOH/THF (1/1, 100), K₂CO₃ (1.97 g, 14.3 mmol) was added. The reaction mixture was agitated for 4 h at ambient temperature. Water (100 ml) was added and the aqueous phase was extracted with CH₂Cl₂ (2×100 ml) and with AcOEt (1×100 ml). The combined organic phases were washed with brine (1×100 ml), dried on Na₂SO₄, filtered and concentrated under reduced pressure. The crude was purified by chromatography (150 g SiO₂, petroleum ether/ethyl acetate, 6:4) yielding compound 2 (3.90 g, 91%, mp: 192.3-195.0° C.) with a solid white appearance.

IR ATR: 3385. 1698, 1682.

¹H NMR (CDCl₃): 3.63-3.50 (1H, m, H₃); 2.50 (1H, t, J=9 Hz, H₁₇); 2.30-1.95 (2H, m); 2.09 (3H, s, H21); 1.90-1.55 (8H, m); 1.50-0.75 (11H, m); 0.79 (3H, s, Me); 0.66 (1H, td, J=3.9-12.0 Hz); 0.58 (3H, s, Me).

¹³C NMR (CDCl₃): 209.7 (C═O); 71.2 (CH, C₃); 63. (CH); 567 (CH); 54.2 (CH); 44.8 (CH); 44.2 (C); 39.2 (CH₂); 38.1 (CH₂); 37.0 (CH₂); 35.5 (CH+C); 32.0 (CH₂); 31.5 (CH₃); 31.4 (CH₂); 28.6 (CH₂); 24.4 (CH₂); 22.8 (CH₂); 21.2 (CH₂); 13.4 (CH₃); 12.3 (CH₃).

### Example 3: Synthesis of the 1-((3S.5S.10S.13S.17S)-3-(allyloxy)-10.13-dimethylhexadecahydro-1H-cyclopenta[alphenanthren-17-yl)ethanone BR297

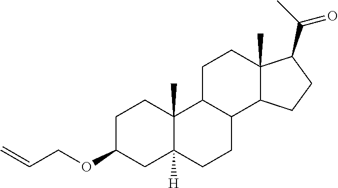 [SMILES: C=CCO[C@H]1CC[C@]2(C)C3CC[C@@]4(C)C(CC[C@@H]4C(C)=O)C3CC[C@H]2C1]

N,N-diisopropylethylamine (5.10 ml, 29.5 mmol) was added to a solution of compound 2 (1.85 g, 5.8 mmol) in DMF (30 ml) and the reaction mixture was agitated under reflux for 30 min. Allyl bromide (1.50 ml, 17.3 mmol) was added at ambient temperature and the reaction mixture was heated under reflux for 6 h30. The solvent was evaporated under reduced pressure. The residue was dissolved in AcOEt (20 ml) and washed in water (2×20 ml). The combined organic phases were extracted with AcOEt (2×20 ml). The combined organic phases were washed with brine (1×20 ml), dried on Na₂SO₄, filtered and concentrated under reduced pressure. The crude was purified by flash chromatography (100 g SiO₂, petroleum ether/ethyl acetate, 6:4), yielding BR297 (1.25 g, 60%, F: 106.2-107.3° C.) with a solid white appearance.

IR ATR: 1704.

¹H NMR (CDCl₃): 5.91 (1H, ddt, J=5.7-10.5-17.1 Hz); 5.25 (1H, dq, J=1.5-17.1 Hz); 5.13 (1H, dq, J=1.5-10.5); 4.00 (2H, d, J=5.7 Hz, CH₂O); 3.25 (1H, tt, J=4.7-11.1 Hz, H₃); 2.51 (1H, t, J=6 Hz, H17); 2.30-0.55 (22H, m); 2.10 (3H, s, OAc); 0.79 (3H, s, Me); 0.59 (3H, s, Me).

¹³C NMR (CDCl₃): 209.8 (C═O); 135.6 (C═CH); 116.4 (C═CH₂); 77.3 (CH, C₃); 58.9 (CH₂O); 63.9 (CH); 56.7 (CH); 54.3 (CH); 44.8 (CH); 44.3 (C); 39.1 (CH₂); 37.0 (CH₂); 35.8 (C); 35.7 (CH); 35.5 (CH₂); 32.0 (CH₂); 31.5 (CH₃); 28.7 (CH₂); 28.2 (CH₂); 22.8 (CH₂); 21.2 (CH₂); 13.4 (CH₃); 12.3 (CH₃).

### Example 4: Synthesis of (3R,5S,10S,13S,17S)-17-acetyl-10,13-dimethylhexadecahydro-1H-cyclopenta[alphenanthren-3-yl benzoate 3

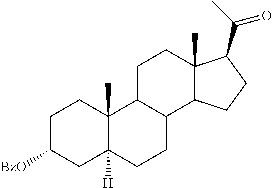

To a solution of compound 2 (1.95 g, 6.1 mmol) in THF (80 ml), benzoic acid (1.12 g, 9.2 mmol), triphenylphosphine (2.40 g, 9.1 mmol) and diisopropylazodicarboxylate (1.80 ml, 9.1 mmol) were added. The reaction mixture was agitated for one night at ambient temperature. AcOEt (50 ml) was added and the organic phase was washed with a solution of saturated NaHCO₃ (2×100 ml). The combined aqueous phases were extracted with AcOEt (2×80 ml). The combined organic phases were washed with brine (1×100 ml), dried by Na₂SO₄, filtered and concentrated under reduced pressure. The crude was purified by chromatography (250 g SiO₂, petroleum ether/ethyl acetate, 9:1), yielding compound 3 (2.32 g, 90%, F: 111-113.1° C.) with a solid white appearance.

IR ATR: 1703. 1449.

¹H NMR (CDCl₃): 8.06 (2H, dd, J=0.9-8.1 Hz, H_{Ar}); 7.55 (1H, t, J=7.5 Hz, H_{Ar}); 7.46 (2H, t, J=7.5 Hz, H_{Ar}); 5.28 (1H, s, H₃); 2.52 (1H, t, J=9 Hz, Hi₇); 2.30-0.80 (22H, m); 2.12 (3H, s, H₂i); 0.85 (3H, s, Me); 0.62 (3H, s, Me).

¹³C NMR (CDCl₃): 209.7 (C═O, C₂₀); 165.9 (C═O, OBz); 132.7 (CH, C_{Ar}); 131.2 (C, C_{Ar}); 129.5 (2CH, C_{Ar}); 128.3 (2CH, C_{Ar}); 70.7 (CH, C₃); 63.9 (CH); 56.7 (CH); 54.2 (CH); 44.3 (C); 40.4 (CH); 39.1 (CH₂); 35.9 (C); 35.5 (CH); 33. (CH₂); 33.0 (CH₂); 31.9 (CH₂); 31.5 (CH₃); 28.3 (CH₂); 26.3 (CH₂); 24.4 (CH₂); 22.8 (CH₂); 20.8 (CH₂); 13.5 (CH₃); 11.4 (CH₃).

### Example 5: Synthesis of 1-((3R,5S,10S,13S,17S)-3-hydroxy-10,13-dimethylhexadecahydro-1H-cyclopenta[alphenanthren-17-yl)ethanone (or allopregnanolone) 4

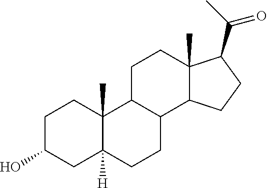 [SMILES: CC(=O)[C@H]1CCC2C3CC[C@H]4C[C@H](O)CC[C@]4(C)C3CC[C@@]21C]

To a solution of compound 3 (390 mg, 0.88 mmol) in MeOH (10 ml), KOH (298 mg, 5.32 mmol) was added. The reaction mixture was heated under reflux for 2 h30. After cooling at ambient temperature, water (20 ml) was added. The aqueous phase was extracted with CH₂Cl₃ (3×20 ml). The combined organic phases were dried on Na₂SO₄, filtered and concentrated under reduced pressure. The crude was purified by chromatography (5 g SiO₂, petroleum ether/ethyl acetate, 7:3), yielding compound 4 (223 mg, 80%, F: 161.7-162.8° C.) with a white solid appearance.

IR (ATR): 3272, 1703.

¹H NMR (CDCl₃): 4.04 (1H, m, H₃); 2.58 (1H, t, J=6 Hz, H₁₇); 2.20-1.90 (1H, m); 2.10 (3H, s, OAc); 1.97 (1H, dt, J=2.7-11.7 Hz); 1.80-0.65 (21H, m); 0.77 (3H, s, Me); 0.59 (3H, s, Me).

¹³C NMR (CDCl₃): 209.8 (C═O); 66.5 (CH, C₃); 63.8 (CH); 56.7 (CH); 54.2 (CH); 44.3 (C); 39.1 (CH); 36.1 (C); 35.8 (CH₂); 35.5 (CH); 32.2 (CH₂); 1.9 (CH₂); 31.5 (CH₃); 29.0 (CH₂); 28.5 (CH₂); 24.4 (CH₂); 22.8 (CH₂); 20.9 (CH₂); 13.5 (CH₃); 11.2 (CH₃).

### Example 6: Synthesis of (3R,5S,10S,13S,17S)-10,13-dimethyl-17-(2-methyl-1,3-dioxolan-2-yl)hexadecahydro-1H-cyclopenta[alphenanthren-3-ol) 5

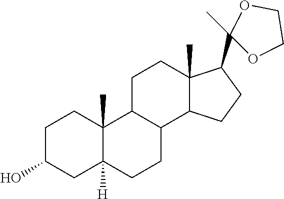 [SMILES: CC1([C@H]2CCC3C4CC[C@H]5C[C@H](O)CC[C@]5(C)C4CC[C@@]32C)OCCO1]

To a solution of compound 4 (221 mg, 0.69 mmol) in toluene (15 ml), ethylene glycol (0.60 ml, 10.92 mmol) and pyridinium p-toluenesulfonate (in catalytic quantity) were added. The reaction mixture was heated under reflux with a Dean Stark device for 3 h and the solvent was evaporated under reduced pressure. The residue was dissolved in CH₂Cl₂ (30 ml) and washed in water (2×20 ml). The combined aqueous phases were extracted with CH₂Cl₂ (2×20 ml). The combined organic phases were dried on Na₂SO, filtered and concentrated under reduced pressure. The crude was purified by flash chromatography (10 g SiO₂, petroleum ether/ethyl acetate, 9:1) yielding compound 5 (173 mg, 69%) with a white amorphous solid appearance.

IR ATR: 3281. 1705.

¹H NMR (CfiDfi): 3.78 (1H, m, H-3β); 3.65-3.45 (4H, m, dioxolane); 2.13 (1H, dt, J=3.3-12 Hz); 2.00-1.70 (3H, m); 1.70-0.60 (17H, m); 1.33 (3H, s, H-21); 0.96 (3H, s, Me); 0.70 (3H, s, Me).

### Example 7: Synthesis of 2-((3R,5S,10S,13S,17S)-3-(allyloxy)-10,13-dimethylhexadecahydro-1H-cyclopenta[alphenanthren-17-yl)-2-methyl-1,3-dioxolane 6

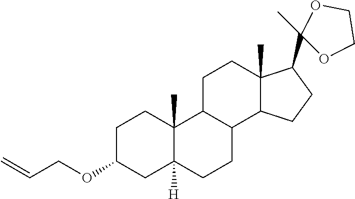 [SMILES: C=CCO[C@@H]1CC[C@]2(C)C3CC[C@@]4(C)C(CC[C@@H]4C4(C)OCCO4)C3CC[C@H]2C1]

To a solution of compound 5 (472 mg, 1.32 mmol) in THF (60 ml), NaH (327 mg, 13.2 mmol) and allyl bromide (1.15 ml, 13.2 mmol) were added. The reaction mixture was heated at reflux for 20 h. After cooling at ambient temperature, water (40 ml) was slowly added. The aqueous phase was extracted with AcOEt (3×40 ml). The combined organic phases were washed with brine (1×40 ml), dried on Na₂SO₄, filtered and concentrated under reduced pressure. The crude was purified by flash chromatography (10 g SiO₂, petroleum ether/ethyl acetate, 95:5), yielding compound 6 (440 mg, 83%, F: 86.2-87.4° C.), having a white solid appearance.

IR (ATR): 1447, 1369.

¹H NMR (CfiDfi): 6.02-5.86 (1H, m, C═CH); 5.32 (1H, dq, J=1.5-17.1 Hz, C═CH₂); 5.06 (1H, dq, J=1.2-10.2 Hz, C═CH₂); 3.85 (2H, d, J=4.8 Hz, OCH₂); 3.65-3.35 (5H, m, dioxolane and H₃); 2.14 (1H, dt, J=3.1-12.2 Hz); 2.00-0.60 (23H, m); 1.32 (3H, s, H₂i); 0.96 (3H, s, Me); 0.76 (3H, s, Me).

¹³C NMR (CfiDfi): 136.9 (CH, C═CH); 115.6 (CH₂, C═CH₂); 112.3 (C, C₂₀); 74.1 (CH, C₃); 69.4 (CH₂, OCH₂); 65.5 (CH₂, dioxolane); 63.6 (CH₂, dioxolane); 59.3 (CH); 57.0 (CH); 54.8 (CH); 42.7 (C); 40.5 (CH); 40.2 (CH₂); 36.6 (C); 35.7 (CH); 34.0 (CH₂); 33.5 (CH₂); 32.7 (CH₂); 29.5 (CH₂); 26.6 (CH₂); 25.2 (CH₃); 24.4 (CH₂); 23.8 (CH₂); 21.4 (CH₂); 13.9 (CH₃); 12.0 (CH₃).

### Example 8: Synthesis of 1-((3R,5S,10S,13S,17S)-3-(allyloxy)-10,13-dimethylhexadecahydro-1H-cyclopenta[alphenanthren-17-yl)ethanone BR351

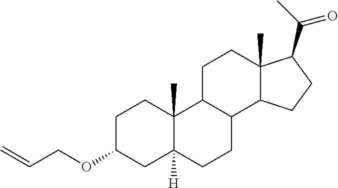 [SMILES: C=CCO[C@@H]1CC[C@]2(C)C3CC[C@@]4(C)C(CC[C@@H]4C(C)=O)C3CC[C@H]2C1]

To a suspension of SiO₂ (5 g) in CH₂Cl₂ (5 ml), 20 drops of a saturated oxalic acid solution were added. The mixture was agitated for 5 minutes and a solution of compound 6 (92 mg, 0.23 mmol) in CH₂Cl₂ (5 ml) was added. The reaction mixture was agitated for 50 minutes at ambient temperature. The silica gel was filtered and washed with CH₂Cl₂ (30 ml) and AcOEt (30 ml). The solvents were evaporated under reduced pressure. The crude was purified by chromatography (5 g SiO₂, petroleum ether/ethyl acetate, 9:1), yielding BR351 (77 mg, 93%, F: 46.5-48.1° C.) having a white solid appearance.

IR ATR: 1701, 1643.

¹H NMR (CfiDfi): 6.10-5.90 (1H, m, C═CH); 5.40-5.30 (1H, m, C═CH₂); 5.15-5.10 (1H, m, C═CH₂); 3.85-3.80 (2H, m, CH₂O); 3.44 (1H, t, J=2.7 Hz, H₃); 2.13 (1H, t, J=6.3 Hz, H₁₇); 1.90-0.50 (25H, m); 0.69 (3H, s, Me); 0.59 (3H, s, Me).

¹³C NMR (CfiDfi): 209.0 (C═O); 136.1 (CH, C═CH); 114.9 (CH₂, CH═CH₂); 73.3 (CH, C₃); 68.7 (CH₂, OCH₂); 63.4 (CH); 65.3 (CH); 53.9 (CH); 39.4 (CH); 38.8 (CH₂); 35.2 (CH₃); 33.2 (CH₂); 32.7 (CH₂); 31.9 (CH₂); 28.6 (CH₂); 25.8 (CH₂); 24.2 (CH₂); 22.8 (CH₂); 20.7 (CH₂); 13.2 (CH₃); 11.2 (CH₃).

### Example 9: Synthesis of (3S,5S,10S,13S)-17-acetyl-10,13-dimethyl-12-oxo-2,3,4,5,6,7,8,9,10,11,12,13,14,15-tetradecahydro-1H-cyclopenta[alphenanthren-3-yl acetate 7

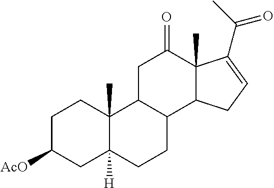 [SMILES: CC(=O)O[C@H]1CC[C@]2(C)C3CC(=O)[C@]4(C)C(C(C)=O)=CCC4C3CC[C@H]2C1]

To a solution of purified hecogenin acetate (5.02 g, 10.61 mmol) in Ac₂O (80 ml), NH₄Cl (0.60 g, 11.21 mmol) and pyridine (1 ml, 12.36 mmol) were added. The reaction mixture was heated at reflux for 15 hours. The obtained orange solution was cooled at 0° C. and a solution of CrO₃ (2.00 g, 20.00 mmol) and water (60 ml) and AcOH (6 ml) was added drop by drop for more than 15 minutes. The black solution was agitated for 1 h30 at ambient temperature, then diluted with water (100 ml) and extracted with Et₂O (3×75 ml). The organic phases were washed with saturated NaHCO₃ (1×100 ml), with brine (1×100 ml), dried on Na₂SO₄, filtered and concentrated under reduced pressure. The green residue obtained was dissolved in AcOH (50 ml) and was heated for 1 h at reflux. The brown residue obtained after the concentration was dissolved in CH₂Cl₂ (50 ml) and washed with water (2×50 ml), with a solution of saturated NaHCO₃ (1×50 ml), dried on Na₂SO₄, filtered and concentrated under reduced pressure. The crude was purified by chromatography (150 g SiO₂, petroleum ether/ethyl acetate, 8:2), yielding compound 7 (2.66 g, 67%, F: 155.2-157.7° C.) having a solid white appearance.

IR ATR: 1721, 1710, 1672.

¹H NMR (CDCl₃): 6.60 (dd, 1H, J=1.8-3 Hz, H-16); 4.67 (m, 1H, H-3a); 2.58 (t, 1H, J=13.2 Hz, H-11); 2.43 (ddd, 1H, J=3.3-6.9-16.8 Hz, H-11); 2.32 (s, 3H, H-21); 2.30-2.10 (m, 1H); 2.25 (dd, 1H, J=5.4-13.2 Hz); 2.05-1.90 (1H, m); 2.02 (s, 3H, CH₃, Ac); 1.95 (dd, 1H, J=3.4-11.4 Hz); 1.88-1.25 (m, 7H) 1.32 (s, 3H, CH₃); 1.25-1.00 (m, 5H); 0.95 (s, 3H, CH₃).

¹³C NMR (CDCl₃): 209.7 (C═O, C-12); 196.3 (C═O, C-20); 170.5 (C═O, OAc); 150.6 (C, C-17); 142.4 (CH, C-16); 73.1 (CH, C-3); 61.1 (C); 56.7 (CH), 55.9 (CH); 44.6 (CH); 38.0 (CH₂); 36.3 (C); 36.1 (CH₂); 33.7 (CH₂); 33.4 (CH); 31.6 (CH₂); 31.4 (CH₂); 28.2 (CH₂); 27.4 (CH₃); 27.2 (CH₂); 21.4 (CH₃); 16.5 (CH₃); 11.8 (CH₃).

HRMS (ESI) m/z: C₂₃H₃₃O₄ [M+H⁺] calculated: 373.2373, found: 373.2384.

### Example 10: Synthesis of (3S,5S,10S,13S,17S)-17-acetyl-10,13-dimethyl-12-oxohexadecahydro-1-cyclopenta[al phenanthren-3-yl acetate 8

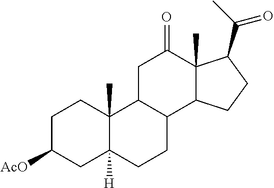 [SMILES: CC(=O)O[C@H]1CC[C@]2(C)C3CC(=O)[C@@]4(C)C(CC[C@@H]4C(C)=O)C3CC[C@H]2C1]

To a solution of compound 7 (2.78 g, 7.47 mmol) in EtOH (70 ml), Pd/C at 10% (0.33 g) was added. The reaction mixture was ventilated with H₂ (vacuum/H₂) and was agitated vigorously under H until absorption of 1.1 eq of H₂ (195 ml, 7.8 mmol). The reaction mixture was ventilated with argon (vacuum/Argon), the catalyst was filtered on Celite and washed with EtOH (50 ml), AcOEt (50 ml), CH₂Cl₂ (50 ml), and concentrated under reduced pressure. The obtained white solid was purified by chromatography (100 g SiO₂, petroleum ether/ethyl acetate, 8:2) yielding compound 8 (2.257 g, 81%, F: 189-192.6° C.) having the appearance of a white solid.

IR (ATR): 1723. 1699.

¹H NMR (CDCl₃): 4.68 (tt, 1H, J=4.9-11.2 Hz, H-3a); 3.30 (t, 1H, J=9.3 Hz, H-17a); 2.48 (t, 1H, J=13.2 Hz, H-11); 2.30-2.05 (m, 2H); 2.26 (s, 3H, CH₃, Ac); 2.02 (s, 3H, CH₃, Ac); 1.90-0.95 (15H, m); 0.94 (s, 3H, CH₃); 0.91 (s, 3H, CH₃).

¹³C NMR (CDCl₃): 212.7 (C═O, C-12); 209.1 (C═O, C-20); 169.0 (C═O; OAc); 72.5 (CH, C-3); 57.5 (C, C-13), 56.7 (CH, C-17); 55.5 (CH); 53.6 (CH); 43.8 (CH); 37.4 (CH₂); 35.7 (CH₂); 35.5 (C); 34.1 (CH₂); 33.1 (CH₂); 30.7 (CH); 30.6 (CH₃); 27.5 (CH₂); 26.5 (CH₂); 23.4 (CH₂); 21.9 (CH₂), 20.7 (CH₃); 12.9 (CH₃); 11.2 (CH₃).

HRMS (ESI) m/z: C₂₃ĤO₄ [M+H⁺] calculated: 375.2530, found: 375.2547.

### Example 11: Synthesis of (3S,5S,10S,13S,17S)-17-acetyl-3-hydroxy-10,13-dimethyltetradecahydro-1H-cyclopenta[alphenanthren-12(2H)-one BR053

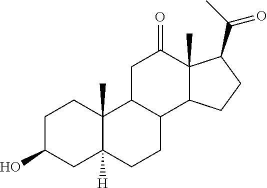 [SMILES: CC(=O)[C@H]1CCC2C3CC[C@H]4C[C@@H](O)CC[C@]4(C)C3CC(=O)[C@@]21C]

To a solution of compound 8 (384 mg, 1.03 mmol) in a mixture of MeOH/THF (1/1, 10 ml), K₂CO₃ (583 mg, 4.22 mmol) was added. The reaction mixture was agitated for 5 h at ambient temperature. Water (10 ml) was added. The aqueous phase was extracted with CH₂Cl₂ (2×20 ml) and with AcOEt (1×20 ml). The combined organic phases were washed with brine (1×20 ml), dried on Na₂SO₄, filtered and concentrated under reduced pressure. The crude was purified by chromatography (10 g SiO₂, petroleum ether/ethyl acetate, 6:4), yielding compound BR053 (317 mg, 93%, F: 183.9-185.6° C.) having the appearance of a white solid.

IR ATR: 3389. 1712, 1697.

¹H NMR (CDCl₃): 3.60 (tt, 1H, J=4.9-11.0 Hz, H-3a); 3.31 (t, 1H, J=9.3 Hz, H-17a); 2.49 (t, 1H, J=13.2 Hz, H-11); 2.40-2.10 (m, 2H); 2.26 (s, 3H, CH₃, H-21); 1.95-0.70 (m, 18H); 0.95 (s, 3H, CH₃); 0.90 (s, 3H, CH₃).

¹³C NMR (CDCl₃): 213.5 (C═O); 209.8 (C═O); 70.9 (CH, C-3); 58.2 (C, C-13); 57.5 (CH); 56.4 (CH); 54.3 (CH); 44.6 (CH); 38.1 (CH₂); 37.8 (CH₂); 36.6 (CH₂); 36.2 (C, C-10); 34.8 (CH₃); 31.4 (2CH₂); 28.3 (CH₂); 24.1 (CH₂); 22.6 (CH₂); 13.5 (CH₃); 11.9 (CH₃).

### Example 12: Synthesis of (3R,5S,10S,13S,17S)-17-acetyl-10,13-dimethyl-12-oxohexadecahydro-1H-cyclopenta[alphenanthren-3-yl benzoate 9

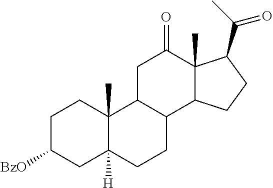

To a solution of compound 8 (2.11 g, 6.35 mmol) in THF (80 ml), benzoic acid (1.17 g, 9.5 mmol), triphenylphosphine (2.52 g, 9.5 mmol) and diisopropylazodicarboxylate (1.90 ml, 9.5 mmol) were added. The reaction mixture was agitated for 15 h at ambient temperature. AcOEt (50 ml) was added and the organic phase was washed with a solution of saturated NaHCO₃ (2×100 ml). The combined aqueous phases were extracted with AcOEt (2×80 ml). The combined organic phases were washed with brine (1×100 ml), dried on Na₂SO₄, filtered and concentrated under reduced pressure. The crude was purified by column chromatography (250 g SiO₂, petroleum ether/ethyl acetate, 9:1), yielding compound 9 (2.56 g, 92%, F: 138.2-141.1° C.) having the appearance of a white solid.

IR ATR: 1702.

¹H NMR (CDCl₃): 8.02 (dd, H, J=0.9-8.1 Hz, H—Ar); 7.54 (t, 1H, J=7.2 Hz, H—Ar); 7.44 (t, 2H, J=7.2 Hz, H—Ar); 5.29 (s, 1H, H-3β); 3.32 (t, 1H, J=9.6 Hz, H-17a); 2.50 (t, 1H, J=13.3 Hz, H-11); 2.40-2.10 (m, 1H); 2.30 (dd, 1H, J=4.8-12 Hz, H-11); 2.26 (s, 3H, CH₃, Ac, H-21); 2.00-0.80 (m, 19H); 0.96 (s, 3H, CH₃); 0.93 (s, 3H, CH₃).

¹³C NMR (CDCl₃): 213.6 (C═O); 209.7 (C═O); 165. (COOBz); 132.8 (CH, C—Ar); 130.9 (C, C—Ar); 129.5 (2CH, C—Ar), 128.4 (2CH, C—Ar); 70.1 (CH, C-3); 58.2 (C, C-13); 57.7 (CH); 56.5 (CH); 54.3 (CH); 40.3 (CH); 37.8 (CH₂); 36.6 (C, C-10); 34.8 (CH); 32.9 (CH₂); 32.7 (CH₂); 31.3 (CH₃); 28.0 (CH₂); 26.1 (CH₂); 24.1 (CH₂); 22.6 (CH₂); 13.5 (CH₃); 11.1 (CH₃).

### Example 13: Synthesis of ((3R,5S,10S,13S,17S)-17-acetyl-3-hydroxy-10,13-dimethyltetradecahydro-1H-cyclopenta[alphenanthren-12(2H)-one BR338

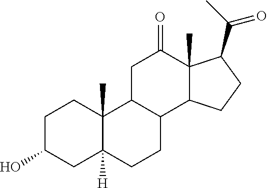 [SMILES: CC(=O)[C@H]1CCC2C3CC[C@H]4C[C@H](O)CC[C@]4(C)C3CC(=O)[C@@]21C]

To a solution of compound 9 (2.44 g, 5.6 mmol) in MeOH (50 ml), KOH (1.41 g, 25.2 mmol) was added. The reaction mixture was heated to reflux for 1 h30. After cooling at ambient temperature, water (40 ml) was added. The aqueous phase was extracted with CH₂Cl₂ (3×40 ml). The combined organic phases were dried on Na₂SO₄, filtered and concentrated under reduced pressure. The crude was purified by chromatography (60 g SiO₂, petroleum ether/ethyl acetate, 8:2), yielding compound BR338 (1.02 g, 55%, F: 193.5-195.6° C.) having the appearance of a white solid.

IR ATR: 3490. 1698.

¹H NMR (CDCl₃): 4.05 (t, 1H, J=2.7 Hz, H-3β); 3.30 (t, 1H, J=8.7 Hz, H-17α); 2.44 (t, 1H, J=13.5 Hz, H-11); 2.40-2.10 (m, 2H); 2.25 (s, 3H, CH₃, Ac, H-21); 1.90-1.10 (m, 18H); 0.93 (s, 3H, CH₃); 0.85 (s, 3H, CH₃).

¹³C NMR (CDCl₃): 213.7 (C═O); 209.9 (C═O); 66.1 (CH, C-3); 58.2 (C, C-13); 57.6 (CH); 56.4 (CH); 54.3 (CH); 38.9 (CH); 37.7 (CH₂); 36.8 (C, C-10); 34.8 (CH); 31.9 (CH₂); 31.4 (CH₂); 28.9 (CH₂); 28.9 (CH₂); 28.2 (CH₂); 24.0 (CH₂); 22.6 (CH₂); 13.6 (CH₃); 10.8 (CH₃).

### Example 14: Evaluation on In Vitro Cell Cultures of Neural Proliferative and Neuroprotective Effects of the Derivatives According to the Invention, or in Compliance with the Invention

The cellular model used to carry out the in vitro experiments is a line of human neuroblastomas called line SH-SY5Y.

### A) Procedure

### A.1. Cell Culture

The human neuroblastoma is a cultivated at 37° C. in a humidity saturated atmosphere, under 5% CO₂ in a culture medium formed by DMEM with an added 10% (v/v) fetal calf serum, 5% (v/v) horse serum, 2 mM glutamax and 1% (v/v) of the penicillin/streptomycin mixture.

### A.2. Microscopic Analysis of the Morphology of the SH-SY5Y Cells

To ensure good quality of the neuroblastomas used, 24 hours before culturing, the SH-SY5Y cells are observed by DMR microscope equipped with a digital camera assisted by a Pentium IV PC computer (Leica, Microsystems, Wetzlan, Germany).

### A.3. Evaluation of the Viability and Density of the Cells: Determination of the Effect of Neurosteroids on Cell Proliferation

The cellular viability and the density (or number of SH-SY5Y cells/cm²) were determined using the MTT reduction assay. The MTT technique is a colorimetric test that measures the reduction of the tetrazolium salt (3-[4,5-dimethylthiazol-2-yl]-2,5-diphenyl tetrazolium bromide) in formazan by the succinate dehydrogenase present in the mitochondria of the living cells. The formazan forms blue crystals. Thus, after solubilization of the crystals, the measurement of the absorbance at 595 nm makes it possible to obtain absorbency values directly proportional to the number of viable cells and the cell density in the culture boxes.

The effects of derivatives BR053, BR338, BR297 and BR351 on cell proliferation were evaluated by comparing the number of viable cells and the cell density in the control culture boxes, treated only with the culture medium described in paragraph A1 above, and the culture boxes exposed to the culture medium with the added BR053, BR338, BR297 or BR351 derivatives. The results are provided in table 1 below.

A.4. Evaluation of the Neuroprotective Effects of the Derivatives According to the Invention or in Compliance with the Invention Against Cell Death Caused by Oxidizing Stress.

The human neuroblastomas were incubated with the culture medium alone or containing the chief inducing agent of oxidizing stress, hydrogen peroxide (H₂O₂), at various concentrations in order to determine the concentrations of H₂O₂ and the incubation times causing the death of a significant percentage of the cells. The neuroprotective properties of the BR053, BR338, BR297 and BR351 derivatives were determined by pretreating the SH-SY5Y cells with the culture medium alone or respectively containing the BR053, BR338, BR297 or BR351 derivative. After pretreatment, each of these categories of cells was integrated for 24 hours or 48 hours with the following solutions: (i) culture medium; (ii) culture medium containing only the BR053, BR338, BR297 or BR351 derivative; (iii) culture medium containing only H₂O₂ at a concentration of 1 mM, which causes a high percentage of cell death; (iv) culture medium containing both H₂O₂ (1 mM) and one of the BR053, BR338, BR297 or BR351 derivatives.

10 μl of a stock solution of tetrazolium, abbreviated MTT, at 3.6 mM was added in each well and the incubation continued in darkness for 5 h at 37° C. After cell lysis, spectrophotometer measurements at 595 nm were carried out to determine the survival and cell density under each of the conditions described above. The results are summarized in table 1 below.

A.5. Study of the Effects of the Derivatives According to the Invention or in Compliance with the Invention on the Type A GABA Channel Receptors (GABAa-R)

These experiments were carried out to verify whether the BR053, BR338, BR297 and BR351 derivatives use the same action mechanisms as allopregnanolone (3alpha,5alpha-THP), in particular the positive allosteric modulation of the GABAa-R, or if these derivatives (BR053, BR338, BR297 and BR351) have differences in their action modes that would prove that they exercise specific activities not evoked by allopregnanolone.

The studies were conducted using an electrophysiological approach. The activity of the cells was recorded using the patch-clamp technique in its so-called whole cell configuration. The mediums used for the recordings have the following composition (in mM): NaCl (32), KCl (2), CaCl₂ (1), MgCl₂ (2), HEPES (10) for the medium bathing the cells and NaCl (132), KCl (2), CaCl₂ (1), MgCl₂ (2), TEA (15), HEPES (10) for the recording pipette, the pH was adjusted in both cases to 7.4 with NaOH. The GABA 10 μM was added into the recording pipette.

After producing a “gigaseal” (Planar patch clamp) between the cellular membrane and the tip of a pipette with an electrical resistance comprised between 4 and 7 MO, the ionic currents were measured using a patch-clamp amplifier (Axopatch B200, Axon Instruments, CA). The acquisition of the signal (acquisition frequency of 10 kHz) was done using an interface card (Digidata 1322A, Axon Instruments, CA) and the pClamp 8 software (Axons Instruments, CA).

The allopregnanolone and the BR053, BR338, BR297 and BR351 derivatives were solubilized in pure ethanol before being diluted in the medium to the desired concentration. The dilution factor was at least equal to 2×10⁴, which makes the final concentration of ethanol in the recording solution remain below 1 μM (a concentration not affecting the activity of the GABAa receptor).

Depending on the experiments, the allopregnanolone or the BR053, BR338, BR297 or BR351 derivatives, at the concentration of 0.25 μM, were either added in the recording pipette at the same time as the GABA, or applied to the cell by perfusion.

The application was done by gravity through a multi-path perfusion system made up of glass tubes each having an inner diameter of 1 mm. The selected path was placed across from the recorded cell at approximately 100 μm. The solution was changed by calibrated rotation of the tubes using a stepping motor thus placing the new selected path across from the cell. Synchronously with that rotation, the solenoid valve controlling the flow of the selected path was also opened and the preceding one closed.

A.6. Revelation of the Non-GABAergic Effects of Derivatives According to the Invention or in Compliance with the Invention

These experiments were carried out under the same recording conditions as the electrophysiological studies described above (A5), with the exception of the GABA, which was not added into the recording pipette.

### B) Results

### B.1. Effects of the Derivatives According to the Invention on Cell Proliferation

The results summarized in the left column of Table 1 below clearly show that the BR297 derivative according to the invention has no effect on the proliferation of human neuroblastomas. This BR297 derivative is therefore completely devoid of any neural proliferative effect. However, the other derivatives stimulate cell proliferation with low effectiveness for BR053, high effectiveness for BR338, and very high effectiveness for BR351.

### B.2. Neuroprotective Effect of the Derivatives According to the Invention Against Cell Death Caused by Oxidizing Stress

As shown by the results provided in the right column of Table 1 below, all of the derivatives according to the invention (BR053, BR338, BR297 and BR351) are capable of protecting human neuroblastomas from the cell death caused by exposure to hydrogen peroxide or H₂O₂, a powerful inducer of oxidizing stress. Owing to their capacity to protect the nervous cells from cell death, these derivatives can therefore be considered neuroprotective compounds. However, it should be noted that the efficacy or power of the neuroprotective action varies depending on the derivatives. Thus, in increasing order of neuroprotective efficacy, the derivatives are: BR338, BR297, BR053 and BR351 (equal to BR053).

TABLE 1
Effective the derivatives according to the invention on
cultures of SH-SY55 human neuroblastomas

| | | Neuroprotective effect against |
|---|---|---|
| | Effect on cell proliferation | cell death caused by oxidizing |
| Compound | (neural proliferative effect) | stress |
| BR053 | + | +++ |
| BR338 | ++ | + |
| BR297 | − | ++ |
| BR351 | +++ | +++ |

The symbols “−”, “+”, “++”, “+++” respectively mean: no effect, low effect, high effect, very high effect.

B.3. Effects of the Derivatives According to the Invention or in Compliance with the Invention on the Type A GABA Channel Receptors (GABAa-R)

As shown by the results provided in Table 2 below, allopregnanolone exerts a positive allosteric effect on the GABAa-R channel. This means that in the presence of GABA (contained in the recording pipette), which activates the GABAa-R on its site to induce an ionic current at the recorded cell, allopregnanolone, by acting on another site of the GABAa-R, potentiates the action of the GABA while increasing the permeability (amplitude) and likelihood of opening of the channel. Interestingly, the comparative analysis of the effects of allopregnanolone and the BR053, BR338, BR297 and BR351 derivatives on the ionic current induced by the GABA via the GABAa-R has revealed action similarities, but also significant differences. In fact, the results show that, unlike allopregnanolone, the BR053 derivative causes a negative allosteric action on the GABAa-R consisting of reducing the amplitude and the likelihood of opening of the channel. The BR338 perfectly mimics the action of the allopregnanolone by exercising, like that neurosteroid, a positive allosteric effect resulting from an increase in the permeability and likelihood of opening of the channel. The BR297 only partially mimics the positive allosteric effect of the allopregnanolone by causing an increase in the likelihood of opening of the GABAa-R channel without increasing its permeability. As for the BR351 derivative, it only temporarily reproduces the positive allosteric effect observed for allopregnanolone (increase of the permeability and likelihood of opening of the GABAa-R channel) and at the end of 1 minute, the BR351 causes desensitization of the GABAa-R channel.

TABLE 2
Effect of the type A GABA receptor channel or GABAa-R

| | |
|---|---|
| Allopregnanolone | Positive allosteric effect |
| BR053 | Negative allosteric effect |
| BR338 | Positive allosteric effect |
| BR297 | Partial positive allosteric effect |
| BR351 | Temporary positive allosteric |
| | effect followed by |
| | desensitization of the channel |

### B.4. Non-GABAergic Effects of the Derivatives According to the Invention

Under the conditions of electrophysiological studies where the GABA was not added in the recording pipette, the BR053 and BR297 derivatives are capable of inducing cellular activity. These results demonstrate that BR053 and BR297 exercise non-GABAergic effects on the nervous cells.

### Example 15: In Vivo Evaluation of the Effects of the Derivatives According to the Invention on the Painful Neuropathy Caused in Rats by Cancer Chemotherapy

### A) Procedure

### A.1. Information on the Animals

Adult male Sprague-Dawley rats weighing between 250 and 300 g were used. They came from the company Janvier (le Genest St. Isle, France). The care and handling of the animals were done in accordance with the guidelines from the Council of the European Community (86/609/EC) and in accordance with the ethical rules of the International Association for the Study of Pain. The protocol used to carry out the in vivo studies was subject to examination and authorization granted to Prof. Mensah-Nyagan's team by the Alsatian Department of the Veterinary Public Health Guide for the Care and Use of Laboratory Animals (Approval number 67-186). The rats were housed under standard conditions with a photoperiod of 12 h day/12 h night, with unlimited food and water. The animals spent one acclimatization week before being integrated into the experimental protocols. Each animal was examined daily to detect any signs of deterioration of its health condition, such as dramatic weight loss, a decrease in appetite during chemotherapy and pharmacological treatment. No suffering was observed in any of the rats included in this study.

### A.2. Pharmacological Substances and Administration Protocols

Vincristine sulfate (VINC), purchased from Sigma-Aldrich (St. Louis, Mo., USA), was dissolved in a physiological saline solution (NaCl 0.9%) at 0.1 mg/ml and stored at 4° C. The saline solution (NaCl 0.9%) is used as excipient. The VINC is injected daily by intraperitoneal injection (i.p.), in 2 cycles of 5 days separated by 2 days off, at a concentration of 0.1 mg/kg/day. The control animals were injected by i.p. (1 ml/kg) with the excipient using a similar protocol.

The allopregnanolone in the derivatives according to the invention (BR053 and BR297) were diluted in a hydroxypropylcellulose (HPC) solution (0.3%) used as excipient. The curative treatment with allopregnanolone, BR053 or BR297 began 3 days after the vincristine cycles. Each of these compounds (allopregnanolone, BR053 or BR297) was administered by the intraperitoneal route at a dose of 4 mg/kg at a rate of one injection every 2 days. Thus, over the total duration of the in vivo experiments (14 days), each animal received 7 injections of excipient, allopregnanolone, BR053 or BR297, based on its group, at a dose of 4 mg/kg. The animals were sacrificed 24 hours after the last behavioral test (28^{th} day) to remove tissues that will be used for later histology and electrophysiology studies.

### A.3. Evaluation of the Mechanical Nociceptive Sensitivity Threshold Using the Von Frey Filament Test

The mechanical nociceptive sensitivity threshold was evaluated in rats placed in individual Plexiglas boxes (30×30×25 cm) mounted on a raised mesh support thereby allowing access to the surface or arch of the back feet. The presence of allodynia (exaggerated response following a normally painful stimulation) was measured using a series of calibrated von Frey filaments (1, 2, 4, 6, 8, 10, 15, 26, 100, 180 and 300 g; Stoelting, Wood Dale, Ill., USA), which were applied on the sole of the back foot of the animal with increasing force until the filament twisted. The filament was applied for a period of 1 to 2 seconds and the procedure was repeated 5 times at 4-5 second intervals. The withdrawal threshold of the foot was calculated by taking the average of 10 repeated stimuli (in grams) that caused a reflex to withdraw the foot. Only vigorous and fast withdrawals of the foot were considered positive. In the experiments, the naïve animals never responded to the 4 g filament and responded 25-30% of the time to the 15 g filament. The observation of responses for the 4 g filament after administering a treatment revealed a mechanical allodynia and the increase in the responses to the 15 g filament indicates mechanical hyperalgesia.

### A.4. Evaluation of Thermal Allodynia to Cold Using the Acetone Test

The thermal nociceptive sensitivity threshold to cold was measured in rats placed in individual Plexiglas boxes (30×30×25 cm) mounted on a raised mesh support so as to allow access to the sole of the animal's foot. 50 μL of acetone was deposited on the animal's foot, and the rats were observed for 20 seconds. Six measurements (3 per foot) were performed with a latency of 5 minutes between each stimulation. The average latency for withdrawal of the feet (in seconds) was calculated by averaging 6 measurements at each studied point in time. Naïve animals do not respond to the application of acetone. A significant decrease in the latency of withdrawal of the feet indicates thermal allodynia to cold.

### B) Results

Chemotherapy treatment with vincristine induces a clear decrease in the mechanical nociceptive sensitivity threshold. Before treatment with vincristine, the Von Frey filament that causes more than 70% of responses (removal of the foot) is 80 g. After chemotherapy with vincristine, a 20 g filament suffices to produce more than 70% of responses. Furthermore, the animals treated with vincristine develop a clearer mechanical allodynia, since the 4 g filament, which did not cause any response before the treatment, causes more than 30% of responses after treatment with vincristine. A mechanical hyperalgesia also appeared in the animals treated with vincristine, since the 15 g fiber, which only caused 30% of responses before administration of vincristine, causes practically 70% of responses after chemotherapy with vincristine.

Furthermore, the acetone test shows a drastic decrease in the latency for withdrawal of the foot (which drops from 20 to 11 seconds) after treatment with vincristine, which shows a thermal allodynia to cold in the treated animals.

Interestingly, each of the neurosteroids, allopregnanolone or the derivatives according to the invention, BR053 or BR297, used at the dose of 4 mg/kg of body weight, advantageously eliminates the symptoms of mechanical allodynia, mechanical hyperalgesia and thermal allodynia. In fact, administration for 2 weeks (an injection every 2 days) of allopregnanolone, BR053 or BR297 at 4 mg/kg of body weight advantageously restores, in rats suffering from painful neuropathies induced by vincristine, the normal or physiological thresholds for thermal and mechanical nociceptive sensitivities. This reestablishment of the normal thresholds for thermal and mechanical nociceptive sensitivities attests to the fact that the allopregnanolone or the derivatives according to the invention, BR053 and BR297, have caused a complete suppression of the neuropathic pain caused by chemotherapy with vincristine.

In healthy animals not having been treated with vincristine, administration for 2 weeks of allopregnanolone (4 mg/kg of body weight every 2 days) causes a significant anti-nociceptive effect characterized by a clear increase in the mechanical nociceptive sensitivity threshold, which goes from 80 g to 200 g. Unlike allopregnanolone, administration for 2 weeks of the derivatives according to the invention, BR053 or BR297 (4 mg/kg of body weight every 2 days), does not cause any anti-nociceptive effect in the healthy animals not having been treated with vincristine.

### CONCLUSIONS

The derivatives according to the invention as defined above or the derivative of formula BR053 defined above are particularly useful, in neuropathic subjects, as compounds making it possible to definitively eliminate pain owing to their capacity to repair the tissue and functional damage responsible for the neuropathy. Experiments conducted in rats suffering from a painful neuropathy caused by vincristine demonstrate that a two-week treatment with the BR297 compound according to the invention or the BR053 derivative and used at a rate of one intraperitoneal injection every 2 days at a dose of 4 mg per kg of body weight, makes it possible to repair the functional deficits evoked by the vincristine and definitively eliminate neuropathic pain. Additional experimental data show that the definitive analgesia produced by BR297 or BR053 in neuropathic animals is not the result of a simple blockage of the transmission of noxious messages (anti-nociceptive effect) or sedation caused by the BR297 or the BR053. In fact, the comparative analysis of the action of the allopregnanolone, BR297 and BR053 on nociceptive transmission in healthy rats shows that at a dose of 4 mg per kg of body weight, allopregnanolone exerts an anti-nociceptive effect related to its sedative power, while the same dose of BR297 and BR053 does not cause any significant anti-nociceptive action. As a result, the only possibility available to the BR297 and BR053 compounds to induce the complete analgesia consisting of definitively eliminating neuropathic pain in pathological animals remains the prior repair of the tissue and functional damage responsible for the neuropathy.

Furthermore, and particularly interestingly, the derivatives according to the invention may distinguish themselves from one another by their effects on cell proliferation.

Thus, BR297, which is an effective neuroprotective agent not exerting any effect on cell proliferation, is a real cause for hope for the treatment and definitive eradication of the painful neuropathies caused by chemotherapy or radiation therapy for cancer. In fact, BR297 can therefore advantageously be prescribed for patients suffering from neuropathies caused by cancer treatment, irrespective of whether the cancer is hormone dependent.

On the other hand, compounds such as BR053 (which has a low neuroproliferative effect), BR338 and BR351 (which induce strong neuronal proliferation), for example, may be contraindicated in cancer patients, in particular in the case of hormone dependent cancers. Nevertheless, these neurosteroids (BR053, BR338 and BR351) advantageously have strong therapeutic potential for the treatment of neurodegenerative pathologies in subjects who do not also have cancer. In particular, BR351, which has considerable neuroproliferative activity and high efficacy and neuroprotection, is a source of real hope for the treatment of neurodegenerative diseases whereof the eradication requires reactivating the neurogenesis in the adult brain to compensate for neuronal losses in the injured cortical territories and preventive protection against deterioration of the cerebral structures still intact in the patient. The therapeutic strategy based on the use of BR351 may be beneficial for millions of patients suffering from various neurodegenerative pathologies throughout the world.

1. A derivative of allopregnanolone or 1-((3R,5S,10S,13S,17S)-3-hydroxy-10,13-dimethylhexadecahydro-1H-cyclopenta[a]phenanthren-17-yl)ethanone or epiallopregnanolone or 1-((3S,5S,10S,13S,17S)-3-hydroxy-10,13-dimethylhexadecahydro-1H cyclopenta[a]phenanthren-17-yl)ethanone according to the following formula (I): 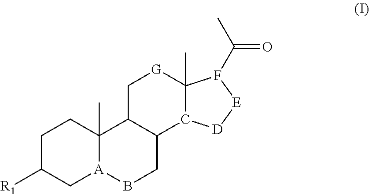
wherein
R¹ is a group chosen among the 3-alpha or 3-beta hydroxy groups, 3-alpha or 3-beta O-allyl groups, 3-alpha or 3-beta O-propargyl groups, 3-alpha or 3-beta O-glycol groups, 3-alpha or 3-beta O-PEG groups, 3-alpha or 3-beta O-glycol-allyl groups and 3-alpha or 3-beta O-PEG-allyl groups,
A is a carbon atom substituted by an atom chosen among 5-H alpha and 5-H beta and B is a methylene group; or
A and B are carbon atoms forming a 5,6-double bond;
C is a carbon atom substituted by an atom chosen among 14-H alpha, 14-H beta, 14-alpha OH group and 14-beta OH and D is a methylene group; or
C and D are carbon atoms forming a 14,15-double bond;
F is a carbon atom substituted by an atom chosen among 17-H alpha and 17-H beta and E is a methylene group or a carbon atom substituted by a group chosen among 16-alkyl-alpha, 16-alkyl-beta, 16-OR²-alpha and 16-OR²-beta, with R² a group chosen among allyl, propargyl, glycol, PEG, glycol-allyl, PEG-allyl, or
F is a carbon atom substituted by a group chosen among 17-alkyl-alpha, 17-alkyl-beta, 17-OR²-alpha and 17-OR²-beta, with R² a group chosen among allyl, propargyl, glycol, PEG, glycol-allyl, PEG-allyl, and E is a methylene group or a carbon atom substituted by group chosen among 16-alkyl-alpha, 16-alkyl-beta, 16-OR²-alpha and 16-OR²-beta, with R² a group chosen among allyl, propargyl, glycol, PEG, glycol-allyl, PEG-allyl, or
E and F are involved in an epoxy cycle or a cyclopropyl and are chosen among 16,17-epoxy-alpha, 16,17-epoxy-beta, 16,17-methylene-alpha and 16,17-methylene-beta; or
E and F are carbon atoms forming a 16,17-double bond;
G is a carbonyl, a methylene or a carbon atom substituted by a 12-OR³-alpha or 12-OR³-beta group with R³ a H atom or a group chosen among the acetyl, alkyl and aryl groups, R³ being able to be chosen among ci-c6-alkyl, benzyl, p-methoxybenzyl, benzoyl, tigloyl, angeloyl, 2,2,2-trichloroethoxycarbonyl, o-aminobenzoyl, nicotinoyl, 2-methylbutyryl, isovaleryl, cinnamoyl, coumaroyl, o-hydroxybenzoyl and anthraniloyl,
excluding (i) the molecule with formula (BR053) below: 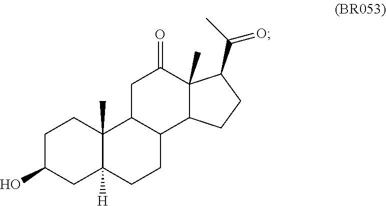 [SMILES: CC(=O)[C@H]1CCC2C3CC[C@H]4C[C@@H](O)CC[C@]4(C)C3CC(=O)[C@@]21C]
and
(ii) the molecule with formula (BR338) below:

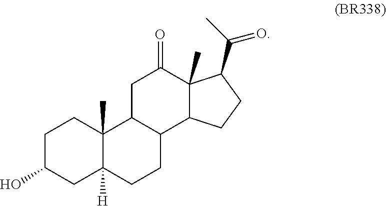 [SMILES: CC(=O)[C@H]1CCC2C3CC[C@H]4C[C@H](O)CC[C@]4(C)C3CC(=O)[C@@]21C]

2. The derivative according to claim 1, wherein
R¹ is a group chosen among the 3-alpha or 3-beta O-allyl groups, the 3-alpha or 3-beta O-propargyl groups, the 3-alpha or 3-beta O-glycol groups, the 3-alpha or 3-beta O-PEG groups, the 3-alpha or 3-beta O-glycol-allyl groups and 3-alpha or 3-beta O-PEG-allyl groups,
A is a carbon atom substituted by an atom chosen among 5-H alpha and 5-H beta and B is a methylene group; or
A and B are carbon atoms forming a 5,6-double bond;
C is a carbon atom substituted by an atom chosen among 14-H alpha, 14-H beta, 14-alpha OH and 14-beta OH and D is a methylene group; or
C and D are carbon atoms forming a 14,15-double bond;
F is a carbon atom substituted by an atom chosen among 17-H alpha and 17-H beta, and E is a methylene group or a carbon atom substituted by a group chosen among 16-alkyl-alpha, 16-alkyl-beta, 16-OR²-alpha and 16-OR²-beta, with R² a group chosen among allyl, propargyl, glycol, PEG, glycol-allyl, PEG-allyl, or
F is a carbon atom substituted by a group chosen among 17-alkyl-alpha, 17-alkyl-beta, 17-OR²-alpha and 17-OR²-beta, with R² a group chosen among allyl, propargyl, glycol, PEG, glycol-allyl, PEG-allyl, and E is a methylene group or a carbon atom substituted by a group chosen among 16-alkyl-alpha, 16-alkyl-beta, 16-OR²-alpha and 16-OR²-beta, with R² a group chosen among allyl, propargyl, glycol, PEG, glycol-allyl, PEG-allyl, or
E and F are involved in an epoxy cycle or a cyclopropyl and are chosen among 16,17-epoxy-alpha, 16,17-epoxy-beta, 16,17-methylene-alpha and 16,17-methylene-beta; or
E and F are carbon atoms forming a 16,17-double bond; and
G is a carbonyl, a methylene or a carbon atom substituted by a 12-OR³ alpha or 12-OR³ beta group with R³ a H atom or a group chosen among the acetyl, alkyl and aryl groups, R³ being able to be chosen among ci-c6-alkyl, benzyl, p-methoxybenzyl, benzoyl, tigloyl, angeloyl, 2,2,2-trichloroethoxycarbonyl, o-aminobenzoyl, nicotinoyl, 2-methylbutyryl, isovaleryl, cinnamoyl, coumaroyl, o-hydroxybenzoyl and anthraniloyl.

3. The derivative according to claim 2, wherein it is a derivative of formula (I) in which R¹ is a group chosen among the 3-beta O-allyl, 3-beta O-propargyl, 3-beta O-glycol, 3-beta O-PEG, 3-beta O-glycol-allyl and 3-beta O-PEG-allyl groups and/or G is a methylene.

4. The derivative according to claim 1 with the following formula (BR297):

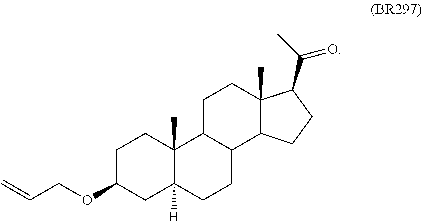 [SMILES: C=CCO[C@H]1CC[C@]2(C)C3CC[C@@]4(C)C(CC[C@@H]4C(C)=O)C3CC[C@H]2C1]

5. The derivative according to claim 1 with the following formula (BR351):

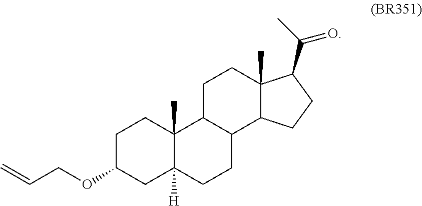 [SMILES: C=CCO[C@@H]1CC[C@]2(C)C3CC[C@@]4(C)C(CC[C@@H]4C(C)=O)C3CC[C@H]2C1]

6. The derivative according to claim 1 for the use thereof as a medicament.

7. The derivative according to claim 1 or derivative with the following formula (BR338): 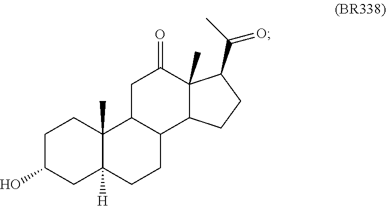 [SMILES: CC(=O)[C@H]1CCC2C3CC[C@H]4C[C@H](O)CC[C@]4(C)C3CC(=O)[C@@]21C]
or the following formula (BR053): 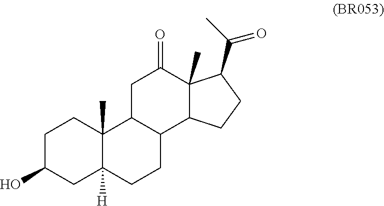 [SMILES: CC(=O)[C@H]1CCC2C3CC[C@H]4C[C@@H](O)CC[C@]4(C)C3CC(=O)[C@@]21C]
for use thereof as a neuroprotective agent and/or as an agent stimulating the proliferation of the nervous cells.

8. The derivative according to claim 7 for the treatment of a neuropathology.

9. The derivative according to claim 8, wherein the neuropathology is chosen among peripheral and central neuropathies, chronic pain, neurodegenerative diseases and sensory motor deficits caused by neuroinflammation.

10. The derivative according to claim 9, wherein the neuropathology is chosen among peripheral and central neuropathies.

11. The derivative according to claim 8, wherein the neuropathology is linked to an allodynia or hyperalgesia.

12. The derivative according to claim 9, wherein the neuropathy is caused by an antineoplastic treatment, in particular a chemotherapeutic treatment using an antineoplastic advantageously chosen among spindle poisons, intercalating agents, topoisomerase DNA inhibitors, tyrosine kinase inhibitors, mTOR inhibitors, anti-angiogenic monoclonal antibodies, inhibitors of DNA synthesis and replication, and inhibitors of the polymerization or depolymerization of microtubules, still more advantageously chosen among vincristine, oxaliplatin, docetaxel, paclitaxel and other derivatives of Taxol®.

13. The derivative according to claim 1 or derivative of the following formula (BR338): 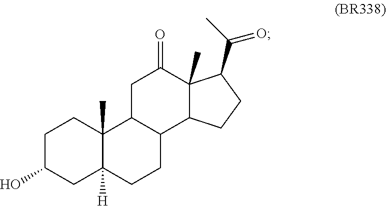 [SMILES: CC(=O)[C@H]1CCC2C3CC[C@H]4C[C@H](O)CC[C@]4(C)C3CC(=O)[C@@]21C]
or the following formula (BR053): 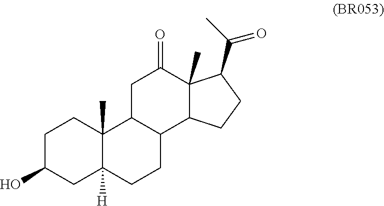 [SMILES: CC(=O)[C@H]1CCC2C3CC[C@H]4C[C@@H](O)CC[C@]4(C)C3CC(=O)[C@@]21C]
for the use thereof as a neuroprotector, in particular a neurorepair agent.

14. The derivative according to claim 1 or derivative of the following formula (BR338): 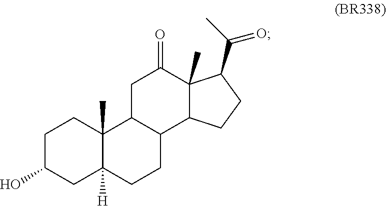 [SMILES: CC(=O)[C@H]1CCC2C3CC[C@H]4C[C@H](O)CC[C@]4(C)C3CC(=O)[C@@]21C]
or the following formula (BR053): 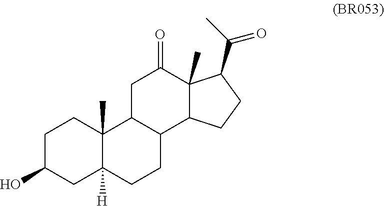 [SMILES: CC(=O)[C@H]1CCC2C3CC[C@H]4C[C@@H](O)CC[C@]4(C)C3CC(=O)[C@@]21C]
excluding the derivative of the following formula (BR297): 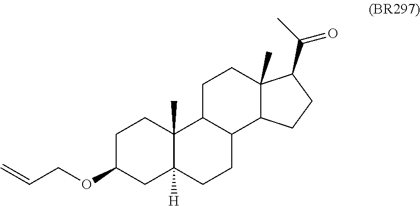 [SMILES: C=CCO[C@H]1CC[C@]2(C)C3CC[C@@]4(C)C(CC[C@@H]4C(C)=O)C3CC[C@H]2C1]
for use thereof as an agent stimulating the proliferation of the nervous cells.

15. The derivative according to claim 4 or derivative of the following formula (BR053): 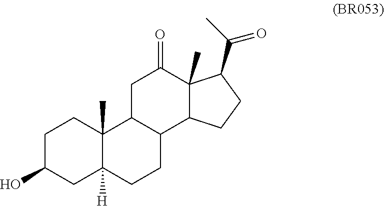 [SMILES: CC(=O)[C@H]1CCC2C3CC[C@H]4C[C@@H](O)CC[C@]4(C)C3CC(=O)[C@@]21C]
for use at a dose of 4 mg of said derivative per kg of body weight, every 2 days, in a patient suffering from a neuropathology as defined according to one of claims 7 to 10.

16. The derivative according to claim 1 or derivative with the following formula (BR338): 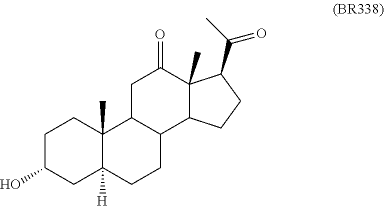 [SMILES: CC(=O)[C@H]1CCC2C3CC[C@H]4C[C@H](O)CC[C@]4(C)C3CC(=O)[C@@]21C]
or the following formula (BR053): 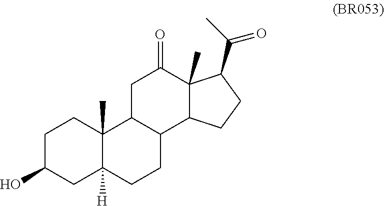 [SMILES: CC(=O)[C@H]1CCC2C3CC[C@H]4C[C@@H](O)CC[C@]4(C)C3CC(=O)[C@@]21C]
to prevent cell death of the neuroblastomas induced by oxidizing stress.
